(19) United States
(12) Patent Application Publication
(10) Pub. No.: US 2016/0051706 A1
(43) Pub. Date: Feb. 25, 2016

(54) PROCESS FOR PRODUCING A COMPLEX OF A LANTHANIDE WITH A MACROCYCLIC LIGAND

(71) Applicant: AGFA HEALTHCARE, Mortsel (BE)

(72) Inventors: Diederik BUFFEL, Mortsel (BE); Paul LEBLANS, Mortsel (BE); Jan VENNEMAN, Mortsel (BE)

(21) Appl. No.: 14/780,684

(22) PCT Filed: Apr. 3, 2014
(86) PCT No.: PCT/EP2014/056670

§ 371 Date: Sep. 28, 2015

(30) Foreign Application Priority Data
Apr. 4, 2013 (EP) 13162339.9

(51) Int. Cl. A61K 49/10

(52) U.S. Cl. CPC A61K 49/108

(57) ABSTRACT

A process for producing a complex of a lanthanide or similar compound with a macrocyclic ligand, wherein the ratio of macrocyclic ligand in free form in relation to the lanthanide or similar compound is equal or more than 0.002% mol/mol, includes the steps of a) measuring the moisture content in a sample of the macrocyclic ligand; and b) mixing an amount G of the lanthanide with an amount X3 of the macrocyclic ligand with the proviso that X3=LG+Lf+M, wherein LG is the amount of macrocyclic ligand necessary for complexing the amount G of lanthanide or similar compound, Lf is an excess amount of the macrocyclic ligand, and M is the amount of moisture present in the amount X3 of the macrocyclic ligand.

### CROSS-REFERENCE TO RELATED APPLICATIONS

This application is a 371 National Stage Application of PCT/EP2014/056670, filed Apr. 3, 2014. This application claims the benefit of European Patent Application No. 13162339.9, filed Apr. 4, 2013, which is herein incorporated by reference in its entirety.

### BACKGROUND OF THE INVENTION

1. Field of the Invention

The present invention relates to a process for producing a complex of a lanthanide or a similar compound with a ligand and which can be used as contrast agent for magnetic resonance imaging.

2. Description of the Related Art

Magnetic resonance imaging (MRI) is a powerful, non-invasive technique used to produce detailed two or three-dimensional anatomical images of tissues in the body. Conventional MRI uses the proton ¹H as its signal source which is highly abundant in tissues and it has the highest sensitivity of all the biologically relevant nuclei.

The contrast, which makes the differentiation of internal structures possible in the image, arises from how the signal decays and is the difference between the resulting signals from two tissue regions. The route by which the protons release the energy they absorbed from the radio-frequency pulse, thus reducing the transverse magnetisation and causing signal decay, is known as relaxation. In MRI two independent relaxation processes occur simultaneously: spin-lattice or longitudinal relaxation characterised by the time constant T₁, and spin-spin or transverse relaxation, characterised by the time constant T₂.

Often, when suitable T₁- or T₂-weighting sequences are used, the natural contrast between two tissues is enough to produce a diagnostically-useful image. However, some conditions do not lead to specific enough changes in the relaxation times of the affected tissue though and then a contrast agent is used to locally change the relaxation times of the diseased tissue, improving the image contrast.

Most contrast agents work by shortening the relaxation times of the water protons in the targeted tissue. T₁ contrast agents are based on paramagnetic metal ion chelates which make the tissue appear brighter on the T₁-weighted image (positive contrast). T₂ contrast agents are usually superparamagnetic iron oxide nanoparticles which create dark spots on the T₂-weighted image (negative contrast). T₁ agents are the most widely used and the majority of these are based on chelates of the gadolinium ion Gd(III).

To be an effective T₁ agent the lanthanide chelate must significantly increase the proton relaxation rates in water. Lanthanide elements are most commonly found in the +3 oxidation state (Ln⁺³), corresponding to the electronic configuration [Xe]6s²4fⁿ. Gadolinium (Gd) is the seventh element in the lanthanide series and has an electronic configuration [Xe]4f⁷. This means that Gd(III) has seven unpaired electrons, making it highly paramagnetic i.e. Gd(III) ions have large permanent magnetic moments (due to electron spin angular momentum), but in the absence of an external magnetic field these are randomly oriented. Due to its large size the Gd(III) and other lanthanides ions typically have a coordination number of nine in its complexes.

Free ions of lanthanides, and in particular gadolinium, are very toxic for the tissues. Indeed, a pathology known as NSF (nephrologic systemic fibrosis, or fibrogenic dermopathy, with very severe effects on human skin), may be at least partly correlated to the existence of free gadolinium ions, i.e. non-complexed gadolinium, in the body. This disease has led to health authorities being alerted with respect to marketed gadolinium-based contrast agents. Briefly, NSF could be associated to the transmetallation of some lanthanide from the complex [lanthanide-chelate] by endogenic ions such as zinc and resulting in unwanted release of free lanthanide ions.

The level of toxicity depends on the strength of the chelating agent, also known as ligand, chelator or sequestering agent to form a complex with the lanthanide ions. Usually these ligands are organic compounds which form two or more separate coordinate bonds with a single central metal ion, in this case, the lanthanide ion, inactivating it and thus reducing or eliminating its toxic effect in the tissues.

Polyaminopolycarboxylic acid compounds are the ligand type of choice because they form exceptionally stable complexes with the Gd(III) ion, which can be explained by a number of reasons. These compounds can be linear (such as pentetic acid or diethylene triamine pentaacetic acid also named as DTPA) or macrocyclic (such as 1,4,7,10-tetraazacyclododecane-1,4,7,10-tetraacetic acid, DOTA). Complexes of macrocyclic ligands are much more kinetically inert and thus, present an exceptionally high solution stability.

Several contrast agents comprising gadolinium as the lanthanide are marketed, Magnevist® (Bayer Healthcare), Multihance® (Bracco), Omniscan® (GE Healthcare), Optimark® (Mallinckrodt Inc.), Dotarem® (Guerbet), Prohance® (Bracco) and Gadovist® (Bayer Healthcare).

EP0270483 discloses contrast agents based on gadolinium with addition of one or more free ligands, such DOTA (1,4,7,10-tetraazacyclododecane-1,4,7,10-tetraacetic acid), EDTA (ethylenediaminetetraacetic acid) or DTPA (diethylene triamine pentaacetic acid) and/or one or more weak metal complexes (presenting relatively low stability constant, such as calcium, magnesium, zinc and iron). The metal ions of the weak metal complex must however be removed via an extra purification step e.g. ultrafiltration to obtain a pharmaceutical formulation which can be directly administrated to the patient.

U.S. Pat. No. 4,647,447 discloses complex salts from the anion of a complexing acid and one or more central ions of an element with an atomic number of 21 to 29, 42, 44 or 57 to 83 and, optionally, also formed from one or more physiologically biocompatible cations of an inorganic and/or organic base or amino acid. This document also discloses the production of contrast agents without isolating the respective complex salts. In this case, to avoid the presence of free toxically active metal ions, such as Gd(III), the chelating step is done by using colour indicators such as xylenol orange by control titrations. If the chelates take up water very quickly, it is not possible to assure that the correct balance between chelate and lanthanide is present in the body, in particular when other ions are present, such Ca²⁺, Zn⁺² and Cu²⁺ which are able to replace the lanthanide ion in some extent, in the complex and thus releasing these to the body. Moreover, no step is disclosed in the method too guarantee that, if an excess of chelate is obtained, the concentration of the free chelate is less than the upper limit of the specifications of the liquid pharmaceutical formulation.

WO2010130814 discloses a process for preparing contrast agents in powder form based on a lanthanide chelate, said powder including an excess of free chelate of 0.002% to 0.4% mol/mol to address the problem of in vivo intolerance of lanthanide chelates related to the presence of free lanthanide ions in the formulation to be administered. Said process includes a step a) of mixing the chelate and the lanthanide wherein the free chelate is in excess in relation to the amount of the lanthanide; b) measuring the amount of free chelate and adjusting said amount to an excess of 0.002 to 0.4% mol/mol in relation to the amount of the lanthanide; and c) precipitating the solution containing the complex obtained with or without the adjustment step in an organic solvent, thus obtaining a powder of chelate-lanthanide, wherein said powder contains an amount of free chelate in excess in relation to the amount of the lanthanide. Besides the fact that it is not possible to assure that the correct balance between chelate and lanthanide is present in the final formulation to be administered, a redissolution of the powder to obtain an injectable pharmaceutical formulation is required.

The undesired release of lanthanide ion is prevented by an amount of excess of ligand. In U.S. Pat. No. 5,876,695 the ligands (ligands L) added in excess are described under the form of an excipient having the formula X[X′, L], where X and X′ are metal ions (especially calcium, sodium, zinc or magnesium) and L is the ligand in excess. These excipients are designed to scavenge free lanthanide. Table 1 of U.S. Pat. No. 7,385,041 illustrates with LD50 values that free macrocyclic ligands (HP-DO3A, DO3A, DOTA) are about at least 10 times more toxic than these macrocycles under the form X[X′, L]. In particular for DOTA, the LD50 is at least about 40 times better for Na₂ [Ca-DOTA] than for free DOTA. The formulation of a macrocyclic ligand administered to the patient should contain, besides the macrocyclic ligand complexed by the lanthanide, an excess of free macrocyclic ligand but in a low range. The free ligand L is not complexed with any metal ions and in particular not under the form of an excipient X[X′, L].

In WO2009103744 it was demonstrated, that a very satisfying tolerance can be obtained when using an amount of excess free macrocyclic chelate at a particular low dose range, and which is not under the form of an excipient X[X′, L]. It was shown that with macrocyclic chelates, and in particular DOTA, results are very advantageous, using a very low excess of free chelate L, so that the pharmaceutical composition administered to the patient contains more specifically between 0.02% and 0.4% and in particular between 0.025% and 0.25 mol % of the free macrocyclic chelate L in relation to the complexed chelate.

As a result, the target values of the free ligand are in a very narrow range. As an injectable product for diagnostic, it is of very high importance that the final pharmaceutical formulation should be manufactured with extremely precise and delicate industrial scale control of the concentrations of free macrocyclic ligands. If this is achieved, no additional purification or isolation steps are required anymore.

By respecting the stoichiometric proportions and by adding an excess of ligand intended not to be complexed with the lanthanide, it is not possible at the industrial scale to achieve sufficient reproducibility in the final pharmaceutical solution of an excess of free DOTA in the target range. This is because of two reasons:
1) the uncertainty of weighing on an industrial scale, which does not make it possible to correctly ensure the ratio (of the order of 1000 to 1) between the chelate and the excess chelate, given the small amount of excess chelate allowed;
2) the rapid uptake from the atmosphere of water by the chelate.

This problem has been solved in WO2009103744 by means of measuring in the liquid pharmaceutical formulation after the complex of the lanthanide with the ligand has been formed, the concentrations of free macrocyclic chelate or of free lanthanide and by adjusting these concentrations by adding additional chelate or lanthanide so as to obtain the desired concentration of an excess of free ligand. This process, however, requires the presence of two measuring methods, one for the lanthanide and one for the ligand in a production installation. Consequently two solutions, one for adjusting the concentration of the lanthanide and another one for the ligand depending on which compound is present in excess, have to be present in the production installation. This increases the complexity of the production process and increase the risk of errors and of introducing microbiological contaminants. Furthermore, it also makes the process more expensive and time consuming. Moreover, adjusting the concentration of the ligand or lanthanide in the final pharmaceutical formulation with a solution of ligand cannot be done very accurately since the ligand in powder form takes up water very quickly which can give considerable weighing errors. To avoid this, the ligand such as DOTA should be kept in very dry conditions or the adjustment of the pharmaceutical formulation should be done in more than one step with intermediate measuring the concentration of the macrocyclic ligand.

It is thus desirable to obtain an optimized process for producing a pharmaceutical liquid formulation on an industrial scale comprising a complex of macrocyclic ligand with a lanthanide, which requires no or only one adjustment of the concentration of the macrocyclic ligand and is fast, accurate and straightforward without the need of a further purification or isolation step(s).

### SUMMARY OF THE INVENTION

The above stated problems are solved by the processes described below. The processes are based on the determination of the moisture content of the macrocyclic ligand which guarantees an excess of macrocyclic ligand after the complex formation of a lanthanide with a macrocyclic ligand on an industrial scale.

Preferred embodiments of the invention are further described below.

Further advantages and embodiments of the present invention will become apparent from the following description and the dependent claims.

### DETAILED DESCRIPTION OF THE PREFERRED EMBODIMENTS

According to a preferred embodiment of the present invention the production of the complex of a lanthanide with a macrocyclic ligand encompasses the following steps:

### A. Measurement of the Moisture Content of the Material Comprising a Macrocyclic Ligand.

In the scope of preferred embodiments of the present invention the macrocyclic ligand is preferably a derivative of tetraaza macrocycles such as 1,4,7,10-tetraazacyclododecane (cyclen), 1,4,7,10-tetrazacyclotridecan (homocyclen) and 1,4,8,11-tetraazacyclotetradecane (cyclam), preferably DOTA (1,4,7,10-tetraazacyclododecane-1,4,7,10-tetraacetic acid), NOTA (1H-1,4,7-Triazonine-1,4,7-triacetic acid, hexahydro), DOTAGA (1,4,7,10-Tetraazacyclododecane-1,4,7,10-tetraacetic acid, α-(2-carboxyethyl)), D03A (1,4,7,10-Tetraazacyclododecane-1,4,7-triacetic acid), DO3A-butrol (1,4,7,10-Tetraazacyclododecane-1,4,7-triacetic acid, 10-[2,3-dihydroxy-1-(hydroxymethyl)propyl]), HP-D03A (1,4,7,10-Tetraazacyclododecane-1,4,7-triacetic acid, 10-(2-hydroxypropyl)) and PCTA (3,6,9,15-Tetraazabicyclo[9.3.1]pentadeca-1(15),11,13-triene-3,6,9-triacetic acid), more preferably DOTA, D03A, HP-D03A and even more preferably DOTA. Most of these macrocyclic ligands take up water from the air very rapidly. The moisture content of macrocyclic ligands can be in the order of 1 to 10 (wt.) % or even higher and lead to a considerable error in weighing the macrocyclic ligand on an industrial scale. Indeed, large batches of macrocyclic ligands cannot be stored in complete moisture free circumstances to avoid water uptake as can be in a lab environment. If no moisture content is taken into account, the amount of macrocyclic ligand in a weighed batch may be too low to complex all the lanthanide and hence the risk is high that free lanthanide in the liquid pharmaceutical formulation will be present or an additional step of adding macrocyclic ligand is required. The macrocyclic ligand including moisture is denoted hereafter as the material comprising the macrocyclic ligand.

In the first step of a method according to a preferred embodiment of the present invention, at least one sample of the material comprising the macrocyclic ligand to be used in production is taken to determine the moisture content. The material comprising the macrocyclic ligand may be homogenised prior to the taking of the sample. Another preferred embodiment is to homogenise the different samples taken prior to the measurement of the moisture. Any known method for the measurement of the moisture content can be used. A preferable method according to the invention is the Karl Fisher method. Another preferable method is measuring the content of the macrocyclic ligand by a suitable analytical technique, e.g. HPLC and calculate the moisture content of the material comprising the macrocyclic ligand. With HPLC, other volatile substances than water such as alcohols are determined. The amount of alcohols can be taken into account based on the information of the Certificate of Analysis of the macrocyclic ligand from the supplier. In order to compensate for the additional amount of moisture which will be taken up by the macrocyclic ligand after the measurement of the moisture content and before the formation of the complex, it is preferred to use an extrapolated moisture content. The extrapolated moisture content can be obtained using a linear or non-linear extrapolation. The moisture content is hereafter presented by ‘MC’ and expressed in % by weight.

The total amount of macrocyclic ligand can be produced by any known method in the art and is preferably homogenised in a container. Said container may be the container where the reaction for producing the ligand occurred, a blender, a homogeniser or any container suitable for homogenising the obtained macrocyclic ligand in the desired amount for a certain batch of formulation.

B. Mixing the Lanthanide with the Macrocyclic Ligand.

### B.1 Lanthanide.

In the scope of preferred embodiments of the present invention, “lanthanides” comprise the fifteen metallic chemical elements with atomic numbers 57 through 71, from lanthanum through lutetium. “Similar compounds” comprise scandium and yttrium. Together with scandium and yttrium, the trivial name “rare earths” is sometimes used to describe all the lanthanides and similar compounds.

Preferred lanthanides and similar compound are Gadolinium (Gd), Yttrium (Y) and Terbium (Tb) and most preferred is Gadolinium.

### B.2 Determination of the Amount of the Material Comprising the Macrocyclic Ligand, X3.

The determination of X3 is done in two steps:

B.2.1. Calculating the Amount X1 of Macrocyclic Ligand Necessary for Obtaining an Excess of Ligand after Complexation.

The total amount of macrocyclic ligand (X1) for a specific batch of complex of lanthanide with a macrocyclic ligand, must be calculated in a way that it is present in the final formulation in an excess (Lf) in relation of the total amount of lanthanide or a similar compound (G), being said amount (Lf) in the range from 0.002% to 0.4% mol in relation of the amount of lanthanide or similar compound (in mol), preferably in the range from 0.02% to 0.3% mol/mol, more preferably in the range from 0.025% to 0.25%, meaning that a small amount of excess ligand should be present after the complex formation. This may ensure that metal traces originated during its production and/or sterilization can be trapped, thus avoiding any possibility of replacement of the lanthanide or similar compound ions in the complex that may result in their release in free form in the contrast agent formulation.

This means that in the final formulation there is an amount LG of macrocyclic ligand forming a complex with lanthanide or similar compound. Hence LG is the amount of macrocyclic ligand necessary for complexing the amount G of lanthanide and an amount in free form Lf, not complexing the lanthanide or similar compound and is thus represented by formula 1:

X1=LG+Lf (1)

### Wherein:

Lf is the amount of macrocyclic ligand in its free form after complex formation, which according to a preferred embodiment of the present invention is in the range set out above in relation of the total amount of the lanthanide or similar compound (G).

The size of the batch of the complex can be small, suitable for laboratory scale i.e. for example of 250 g, 500 g 1000 g, etc. but the advantage of the method is that the method is particularly suited for bigger sizes such as 20 kg, 50 kg, 200 kg, 500 kg, etc., i.e. for pilot and industrial scale.

### B.2.2. Calculation of the Total Amount X3 of Material Comprising a Macrocyclic Ligand for the Production of a Complex of a Macrocyclic Ligand and a Lanthanide.

Based on the result of the measurement of the moisture content (MC) in the macrocyclic ligand as described in section A, it is now possible to calculate or determine the total amount of material comprising the macrocyclic ligand, hereafter denoted as X3 which is required for the production of a batch of the complex. X3 being the sum of X1 and M according to formula 2:

X3=X1+M (2)

Wherein M is the amount of moisture present in the amount of material comprising the macrocyclic ligand (X3) which will be used in the production of the complex of the lanthanide and the macrocyclic ligand. M must be expressed in the same units as the units of Xl. Since MC is the moisture content of X3, MC=M*100/(X1+M).

The total amount of moisture M can be calculated based on X1 and the measured moisture content MC according to formula 3:

M=MC*X1/(100−MC) (3)

Wherein MC is expressed in (wt.) %.

In another embodiment, when a certain amount (Y) of material comprising a macrocyclic ligand is taken as a sample for controlling purposes, ex. quality control, then said amount (Y) may be added to the calculated amount of material in order to compensate the amount that was taken for testing.

If the amount of material comprising the macrocyclic ligand+moisture is introduced into the formulation, the amount of ligand available to form the complex with a lanthanide or similar compound may be not enough when the amount of moisture (M) is not taken into account. Therefore, by calculating X3 it is possible to know the exact amount of material comprising the macrocyclic ligand necessary to add to obtain a certain batch of the complex of a lanthanide with the macrocyclic ligand and avoiding the presence of free lanthanide after complexation and in the final formulation.

Alternatively it is possible to further calculate the amount of material comprising the macrocyclic ligand necessary to fill each unit package when the batch is optionally divided into portions. Thus according formula 4:

X2=X3/N1 (4)

### Wherein,

X2 is the amount in weight of material comprising the macrocyclic ligand with moisture (M) in each unit package; and
N1 is the number of unit packages for one final formulation batch. In another embodiment, the batches of material comprising the macrocyclic ligand may be divided into portions witch are not equal to each other.

In the embodiment where the material comprising a macrocyclic ligand is divided into portions, each unit package is then filled with a homogenised and specifically measured amount (X2) of material under controlled conditions of humidity so that the powder ligand does not further absorb or desorb water during this phase. In consequence, the real content of macrocyclic ligand able to form a complex with a lanthanide or similar compound and of moisture in each unit package is known and thus no further measurements or adjustments are needed; when it is desired to use a certain amount of ligand to produce a batch of formulation it is only necessary to calculate the number of unit packages to achieve the total amount of necessary ligand.

The total amount (X2) of the material of the batch can be divided into portions and then packed. In such case, the amount (N1) of unit packages necessary to pack the material comprising the macrocyclic ligand can be also calculated in function of the total volume of the batch and the size of each unit package.

### B.3. Complex Formation.

The complex formation according to a preferred embodiment of the invention is now described. In one embodiment, the lanthanide or similar compound is added in the form of oxides or salts thereof to the material comprising the macrocyclic ligand to form the complex. In another embodiment, the material comprising the macrocyclic ligand is added as a solution, preferably an aqueous solution, to the lanthanide. Both compounds may also be added simultaneously to the solvent, preferably water. Preferably the macrocyclic ligand is dissolved in a solvent, most preferably water before the lanthanide is added as an oxide or salt. The total amount G of lanthanide or similar compound necessary to form the said complex depends on the amount LG of ligand desired to form a complex, i.e. G should correspond to LG. ‘Corresponding’ means that the amounts are calculated taken the stoichiometry of the complexation reaction into account. The preferred lanthanide or similar compound is gadolinium, terbium or yttrium, most preferable compound is gadolinium. The preferred form of providing gadolinium is in the form of the oxide, i.e. Gd₂O₃.

Another preferred embodiment is to fix the amount G of the lanthanide depending on the batch size of the pharmaceutical formulation to be obtained. Depending on G, the amount of ligand LG, necessary for forming a complex, is then calculated.

Preferably the macrocyclic ligand is dissolved in a solvent, most preferably water before the lanthanide is added. The precise experimental conditions of the mixing are detailed in the examples. Advantageously, the temperature for the complex formation is between 60 and 100° C., and is advantageously about 80° C. The mixture of the lanthanide and the ligand is kept at the elevated temperature for 1 to 4 hours, preferably at least 3 hours. After the complex formation has taken place the obtained solution can be cooled down to room temperature. Moreover, it is understood that the complex formation may be performed in several sub-steps which would be equivalent to an overall complexation step. The sub-steps comprise the addition of the lanthanide or the macrocyclic ligand via multiple steps instead of adding them in one step.

### C. Adjustment of the Amount or Concentration of Macrocyclic Ligand in a Free Form.

According to a preferred embodiment of the present invention, an excess of macrocyclic ligand in solution is obtained. This excess, also called free macrocyclic ligand may be, due to small variations in the process, too high. This means that the concentration of free macrocyclic ligand in solution is then higher than a target level Cₜ. This target level is for example determined by the upper limit of the product specifications related to the concentration of the free macrocyclic ligand of the liquid pharmaceutical formulation. In a preferred embodiment of the present invention an additional adjustment step is therefore provided. According to that embodiment of the invention, the concentration of the free macrocyclic ligand in the solution after formation of the complex is measured. Preferably, a sample from the solution obtained as described in section B.3. is taken and eventually cooled down to room temperature. The measurement can be performed by any suitable analytical method. Suitable methods are detailed in the examples; also HPLC can be used to measure the concentration of the macrocyclic ligand in free form. The concentration of the macrocyclic ligand is then compared with the target value Cₜ. If the concentration is higher than the target value, an amount of lanthanide can be added to the solution. Preferably the solution is therefore kept at an elevated temperature between 60 and 100° C. The amount of required lanthanide, preferably added as a salt or oxide can be added in one step, based on a calculation or in multiple steps. Since the oxide or salts of lanthanides take up much less water than the macrocyclic ligands, the adjustment of the concentration of macrocyclic ligand in the solution by means of adding lanthanide is much more accurate than adjusting an excess of free lanthanide with a solution of macrocyclic ligand. If the lanthanide is added in multiple steps a measurement of the concentration of the macrocyclic ligand in free form can be performed between the multiple steps. Additionally the presence of lanthanide in its free from in the solution can be checked for safety reasons. The solution obtained after the optional adaptation comprises the complex of the lanthanide with the macrocyclic ligand and an excess of free macrocyclic ligand which concentration is within the product specifications of the final pharmaceutical formulation. This solution is now ready to be used in the process of producing a liquid pharmaceutical formulation without the need for additional isolation or purification steps. The analysis of the free lanthanide can be performed by using, for example, a solution of EDTA in the presence of xylenol orange or Arsenazo as indicator. Free gadolinium can be analysed advantageously with a colorimetric method using 0.01 M edetate disodium titration solution in the presence of xylenol orange as an indicator. Titration is then preferably carried out at pH=5 in a sodium acetate/acetic acid buffered solution on a sample of the solution comprising the complex until the colour of the indicator turns from red to yellow.

The intended amount of free ligand (Lf) in the liquid formulation is in the range from 0.002 mol/mol % to 0.4 mol/mol % in relation of the total amount of lanthanide or similar compound (G), preferably is in the range from 0.02 mol/mol % to 0.3 mol/mol %, more preferably in the range from 0.025 mol/mol % to 0.25 mol/mol % in relation to the total amount of lanthanide or similar compound (G).

### D. Process for Producing a Liquid Pharmaceutical Formulation

The remaining components necessary to obtain a liquid pharmaceutical formulation according to a preferred embodiment of the invention and comprising the complex of the lanthanide with the macrocyclic ligand, prepared as described above, are added to the material prepared as described above or optionally to a certain number of unit packages in the desired amounts in function of the different contrast agent formulations. This step may include the addition of the necessary excipients to obtain a formulation with the desired pharmaceutical properties. Examples of such excipients are water, meglumine, hydrochloric acid and/or sodium hydroxide for a pH adjustment.

Examples of excipients added to the complex of a macrocyclic ligand with a lanthanide or similar compound to obtain a liquid pharmaceutical formulation as described above are water for injection, an organic base, hydrochloric acid and/or sodium hydroxide for pH adjustment. The pH is preferably in a range from 4.0 to 8.5 and more preferably in a range from 6.5 to 7.9. The organic base is preferably meglumine.

These excipients may be added to the reaction vessel containing the complex of a macrocyclic ligand and the selected lanthanide or similar compound produced as described above. The macrocyclic ligand is preferably present in the final liquid formulation in a concentration in the range between 0.3 to 0.7 M, preferably in the range between 0.4 to 0.6 M and most preferably at 0.5 M.

Specific embodiments will now be described in detail. The examples are intended to be illustrative and the claims are not limited to the materials, conditions or parameters set forth in the examples.

### EXAMPLES

### Measurements

### Moisture Content in DOTA.

The measurement of the moisture content in DOTA is performed as described in the semi-micro method of Karl Fischer (Fischer, Karl—Pharm. Eur. Section 2.5.12).

### Analysis of DOTA in Free Form (Free DOTA) in Solution.

Solution A: 50 g of sodium acetate was dissolved in 10 ml of glacial acetic acid and the volume was adjusted to 1000.0 ml with water free from carbon dioxide. The obtained solution was adjusted to pH (5±0.05) with 0.1 M sodium hydroxide solution or glacial acetic acid.

Solution B: 50.8 mg of xylene orange was dissolved in water free from carbon dioxide, the volume was adjusted the volume to 100.0 ml with the same solvent. Freshly prepared solutions were used.

Solution C: 3 ml of solution B was added to 30 ml of solution A, the volume of the solution was added to 200.0 ml with water free from carbon dioxide.

0.005 M gadolinium sulphate solution was prepared as follows: 3.735 g of gadolinium sulphate octahydrate was dissolved in water free from carbon dioxide. The volume of solution was adjusted to 1000 ml with the same solvent.

A test solution was prepared as follows: 4.88±0.5 g of meglumine and 50±1 g of hot (70-90° C.) sample of the solution comprising the gadolinium-DOTA complex, were transferred into a 100 ml conical flask, mixed for 5 to 10 minutes at 70° C. to 90° C. and cooled to room temperature. To 2 ml of this solution, 20 ml of water free from carbon dioxide and 10 ml of solution C were added and mixed. The resultant solution was adjusted to pH 5±0.05 with 0.1 M sodium hydroxide solution or glacial acetic acid. The yellow coloration indicates the presence of free DOTA. The solution was titrated with the 0.005 M gadolinium sulphate solution until colour alters to reddish-pink 0.1 ml of 0.005 M gadolinium sulphate solution corresponds to 4.044 mg of DOTA.

### Check for Presence of Free Gadolinium Ions in Solution

4.88±0.5 g meglumine and 50±1 g of hot (70-90° C.) solution comprising the gadolinium-DOTA complex were transferred into a 100-ml conical flask, mixed for 5 to 10 minutes at 70° C. to 90° C. and cooled to room temperature. 2 ml of this solution was added to 20 ml of water free from carbon dioxide and 10 ml of solution C. The resultant solution was adjusted to pH (5±0.05) with 0.1 M sodium hydroxide solution or glacial acetic acid. A red-violet coloration indicates the presence of free gadolinium ions.

### Materials

All reagents used in the following examples were readily available from commercial sources unless otherwise specified:
Gadolinium oxide: Gd₂O₃ from Rhodia
Meglumine: N-methyl-D-glucamine from Merck KGaA, Darmstadt
DOTA: was obtained as described in the unpublished patent application EP13152873.9. The obtained DOTA was stored in drums with a PE bag inside.

### Calculation of X3.

### Preparation of a Batch of Macrocyclic Ligand DOTA for the Production of a 200 L Batch of the Gd-DOTA Complex.

In this section it is explained how the amount X3 of DOTA was calculated for the production of a batch of 200 L of a Gd-DOTA complex at a concentration of 0.5 M (mol/L) taking into account a measured moisture content of the DOTA and a certain Lf value.

In a first step, X1 was calculated. A batch of 200 L of Gd-DOTA complex at a concentration of 0.5 M corresponds with an amount LG of DOTA required for the complex formation of 100 moles or 40.442 kg. For a chosen excess of free DOTA of 0.2% mol/mol Gd, Lf is equal to 0.2 moles or 80.88 g. The amount X1 was calculated according to formula 1 defined above and hence the obtained value of X1 was 40.523 kg.

In a second step, the moisture content MC, expressed as weight percentage, in the sample taken from a DOTA batch was measured by the method described above. The result was 5.0 (wt.) %., which allows to calculate the total amount M of moisture present in the batch of DOTA required for the production of the 200L batch of Gd-DOTA complex, as follows (see formula 3 defined above):

M=MC*X1/(100−MC)=5.0*40.523 kg/(100−5.0)=2.133 kg

X3 was then calculated as X1+M (formula 2 defined above), resulting in a value of 42.656 kg.

### Measuring the Moisture Content of the DOTA.

This section and the next section illustrates how the moisture content in a batch of a macrocyclic ligand changed when the product was exposed to different conditions of relative humidity. The moisture content in the samples was measured using methods known to those skilled in the art. In this example, the measurement of the content of moisture in DOTA was performed as described above.

Two samples of DOTA were kept in conditions of relative humidity (RH)=30+5% at a temperature of Temp=20+2° C. From these samples, small samples were taken at different times (T₀=beginning of the experiment; T₃₀=T₀+30 min; T₉₀=T0+90 min; and T₃₀₀=T₀+300 min) and the moisture content was measured as described above. Each experiment (Exp.) was performed twice and the average of each (Ind.) was determined (Avg.). The results in percentage of the initial weight of DOTA are shown in Table 1.

TABLE 1

| | Moisture content DOTA (wt. %) at a Temp. = | | | | | | | |
|---|---|---|---|---|---|---|---|---|
| | 20 ± 2° C. and RH = 30 ± 5% | | | | | | | |
| | T₀ | | T₃₀ | | T₉₀ | | T₃₀₀ | |
| | Ind. | Avg. | Ind. | Avg. | Ind. | Avg. | Ind. | Avg. |
| Exp. 1 | 4.941 | 4.929 | 4.944 | 4.98 | 5.038 | 5.00 | 5.178 | 5.15 |
| | 4.929 | | 5.020 | | 4.954 | | 5.114 | |
| Exp. 2 | 5.346 | 5.25 | 6.154 | 6.20 | 6.062 | 6.12 | 6.106 | 6.19 |
| | 5.150 | | 6.247 | | 6.170 | | 6.268 | |

These results show that at 30% RH the moisture content in the selected DOTA batch increased in time. These experimental values could be used for extrapolating the moisture content to obtain an extrapolated moisture content.

### Measuring the Moisture Content of the DOTA Ligand in Conditions of Higher RH.

This measurement, as the previous one, illustrated how the moisture content in a batch of a macrocyclic ligand was affected by the environmental relative humidity conditions. Therefore, the previous experiments were repeated in the same conditions but at a RH=75+5%. Again, each experiment (Exp.) was performed twice and the average of each (Ind.) was determined (Avg.). The results in percentage of the initial weight of DOTA are shown in Table 2.

TABLE 2

| | Moisture content DOTA (wt. %) at Temp. = | | | | | | | |
|---|---|---|---|---|---|---|---|---|
| | 20 ± 2° C. and RH = 75 ± 5% | | | | | | | |
| | T₀ | | T₃₀ | | T₉₀ | | T₃₀₀ | |
| | Ind. | Avg. | Ind. | Avg. | Ind. | Avg. | Ind. | Avg. |
| Exp. 3 | 4.941 | 4.94 | 6.734 | 6.71 | 7.475 | 7.34 | 5.914 | 5.86 |
| | 4.929 | | 6.694 | | 7.200 | | 5.813 | |
| Exp. 4 | 5.346 | 5.25 | 6.449 | 6.65 | 6.962 | 6.88 | 4.111 | 4.36 |
| | 5.150 | | 6.851 | | 6.789 | | 4.609 | |

These results showed that the moisture content in DOTA increased with time and with relative humidity of the atmosphere at which the DOTA was stored.

Preparation of a Complex of DOTA with Gadolinium and Adjusting the Concentration of the Free DOTA in Solution.

Two batches, INV01 and INV02 of DOTA were prepared for the production of gadolinium-DOTA complex batches of respectively 50 L and 5 L. The amount X1 of DOTA was calculated in order to obtain an Lf value of respectively 12.97 g for INV01 and 1.30 g for INV02. This corresponds to a free DOTA ratio to gadolinium of 0.124% mol/mol. A target value for the concentration of the free DOTA after the complex formation, Cₜ, was set to 0.045 g/100 ml or 0.128% mol/mol gadolinium, as being the upper limit of the product specification of the liquid pharmaceutical formulation which had to be obtained.

The moisture content of the samples taken from batch INV01 and batch INV02 was determined according to the method as described above.

The results of the measurements, the determined amounts M and the calculated amounts X3 according to formula 3 are reported in Table 3.

TABLE 3

| | INV01 (50 L) | INV02 (5 L) |
|---|---|---|
| Amount X1 of DOTA (kg) | 10.123 | 1.012 |
| Moisture content of DOTA batch | 6.71 | 6.00 |
| (wt. %) | | |
| Total amount of moisture M | 0.727 | 0.065 |
| (kg) | | |
| Amount X3 of DOTA (kg) | 10.85 | 1.077 |
| Amount G of gadolinium oxide | 4.59 | 0.454 |
| (kg) | | |

INV01 was further prepared by weighing DOTA in an amount X3=10.85 kg of DOTA including moisture (see Table 3). This amount of DOTA was stepwise added via a pipe into a tank already containing 35.00 kg of water. Hereafter 4.59 kg of gadolinium oxide was stepwise added via a pipe into the tank containing the water and the DOTA. The moisture content of the gadolinium oxide was 0.2 (wt.) %. The pipes were rinsed with 3.96 kg of water. The reaction mixture was heated to 97° C. and kept at this temperature for 180 minutes. From the solution, samples were taken to check for the presence of free gadolinium and for determining the concentration of free DOTA. The results are reported in Table 4. The solution after using the batch DOTA of INV01 for the complex formation showed no free gadolinium ions.

INV02 was further prepared by weighing DOTA in an amount X3 as reported in Table 3 and added through a funnel into a 6.0 L glass reactor equipped with a stirrer, a thermometer, and a backflow condenser. An amount G of gadolinium oxide (see Table 3) corresponding with LG was added through the same funnel. The moisture content of the gadolinium oxide was 0.2 (wt.) %. 3.9 L of water for injection was added through the same funnel and was washing down the raw material residing from the funnel. The mixture was heated during stirring to a temperature of 95-99° C. The mixture was stirred for 3 hours at this temperature. The stirrer was turned off and the presence of free gadolinium was checked and the concentration of free DOTA in the reaction mixture was determined as described above. The results are reported in Table 4. The reaction mixture after using the batch DOTA of INV02 did not contain free gadolinium ions.

As the concentration of free DOTA in both the solutions after gadolinium-DOTA complex formation using the DOTA batches INV01 and INV02 were exceeding the target value Cₜ of 0.045 g/100 ml of DOTA, additional amounts A of gadolinium oxide were further added to adjust the concentration of free DOTA. The solution is therefore kept at a temperature of 95-99° C. for 95 minutes. The amounts A added were calculated on the basis of formula 5 and are reported in Table 4.

$$A = \frac{V * ( Cd - 0.025 ) * 181.25}{404.42 * 100} \qquad ( 5 )$$

wherein:
V=volume of the solution comprising the gadolinium-DOTA complex in ml.
Cd=concentration in solution of free DOTA in g/100 ml.

The resulting concentrations of free DOTA obtained in the final composition related to INV01 and INV02, showed an excess which was still sufficient to avoid free gadolinium in the final solution and which was lower than the target value Cₜ of 0.045 g/100 ml. Only one adjustment with gadolinium oxide was required to obtain a final composition with the concentration of free DOTA within the specifications. This final composition could now be used for obtaining a liquid pharmaceutical formulation without further isolation or purification steps as described below.

TABLE 4

| | INV01(50 L) | INV02 (5 L) |
|---|---|---|
| Free gadolinium | Below detection limit | Below detection limit |
| Measured concentration | 0.425 (g/100 ml) | 0.259 (g/100 ml) |
| of free DOTA after | 2.102 (% mol/mol Gd) | 1.281 (% mol/mol Gd) |
| complex formation | | |
| Additional amount A of | 89.63 | 5.24 |
| gadolinium oxide (g) | | |
| Free gadolinium after | Below detection limit | Below detection limit |
| addition of gadolinium | | |
| oxide | | |
| Free DOTA concentration | 0.018 g/100 ml | 0.014 g/100 ml |
| in final composition | 0.089 (% mol/mol Gd) | 0.069 (% mol/mol Gd) |
| after addition of | | |
| gadolinium oxide | | |

### Preparation of the Liquid Pharmaceutical Formulation

The final composition prepared with the DOTA batch INV01 was cooled to a temperature between 40° C. and 50° C. and 4.88 kg meglumine was added under agitation. After 40 minutes, the agitation was stopped. The pH of the solution was then measured. 3 g of meglumine was further added to the solution to obtain a pH value of the solution between 7.1 and 7.8. The obtained final liquid pharmaceutical formulation was now ready to be put into vials.

1-13. (canceled)

14. A process for producing a complex of a rare earth with a macrocyclic ligand, wherein a ratio of macrocyclic ligand in free form in relation to the rare earth is equal or more than 0.002% mol/mol, the process comprising the steps of:
measuring a moisture content in a sample of the macrocyclic ligand; and
mixing an amount G of the rare earth with an amount X3 of the macrocyclic ligand such that X3=LG +Lf+M; wherein
LG is an amount of macrocyclic ligand necessary for complexing the amount G of the rare earth;
Lf is an excess amount of the macrocyclic ligand; and
M is an amount of the moisture content present in the amount X3 of the macrocyclic ligand.

15. The process according to claim 14, wherein the ratio of macrocyclic ligand in free form in relation to the rare earth is in the range from 0.002% to 0.4% mol/mol.

16. The process according to claim 14, further comprising the steps of:
measuring a concentration of the macrocyclic ligand in free form; and
if the concentration of the macrocyclic ligand in free form is higher than a target value Cₜ, adding extra rare earth to obtain a ratio of macrocyclic ligand in free form in relation to the rare earth of more than 0.002% mol/mol.

17. The process according to claim 16, wherein the ratio of macrocyclic ligand in free form in relation to the rare earth after adding the extra rare earth is in the range from 0.002% to 0.4% mol/mol.

18. The process according to claim 14, wherein the macrocyclic ligand is DOTA, DO3A, or HP-DO3A.

19. The process according to claim 17, wherein the macrocyclic ligand is DOTA, DO3A, or HP-DO3A.

20. The process according to claim 14, wherein the rare earth is gadolinium, terbium, or yttrium.

21. The process according to claim 14, wherein the rare earth used in the mixing step is gadolinium oxide.

22. The process according to claim 16, wherein the macrocyclic ligand is DOTA and the rare earth used in the mixing step and the adding step is gadolinium oxide.

23. The process according to claim 17, wherein the macrocyclic ligand is DOTA and the rare earth used in the mixing step and the adding step is gadolinium oxide.

24. The process according to claim 14, wherein the macrocyclic ligand is homogenised prior to the measuring step.

25. The process according to claim 14, wherein M is an extrapolated value based on the moisture content obtained in the measuring step.

26. A process of producing a liquid pharmaceutical formulation, the process comprising the steps of:
producing a complex according to the process as defined in claim 14;
adding an organic base to the complex obtained in the step of producing to form a solution; and
optionally adjusting the pH of the solution to a range from 6.5 to 7.9 with hydrochloric acid and/or sodium hydroxide.

27. The process according to claim 26, wherein the organic base is meglumine or a salt thereof, the macrocyclic ligand is DOTA, and the rare earth in the mixing step is gadolinium oxide.

28. A method of magnetic resonance imaging comprising the step of using a liquid pharmaceutical formulation as obtained by the method of claim 27 as a contrast agent for the magnetic resonance imaging.
